(19) United States
(12) Patent Application Publication
(10) Pub. No.: US 2021/0330314 A1
(43) Pub. Date: Oct. 28, 2021

(54) ANCHOR IMPLANTATION SYSTEM

(71) Applicant: Intai Technology Corp., Taichung City (TW)

(72) Inventors: Hui-Ping Huang, TAICHUNG CITY (TW); Ju-Yi Ho, TAICHUNG CITY (TW); Wei-Ren Su, TAICHUNG CITY (TW); Ming-Long Yeh, TAICHUNG CITY (TW); Chen-Hao Chen, TAICHUNG CITY (TW)

(21) Appl. No.: 16/884,646

(22) Filed: May 27, 2020

(30) Foreign Application Priority Data
Apr. 27, 2020 (TW) 109114001

(51) Int. Cl. A61B 17/04

(52) U.S. Cl. CPC A61B 17/0401; A61B 2017/044; A61F 2002/0852; A61B 2017/0445; A61B 2017/0409

(57) ABSTRACT

This invention is related to an anchor implantation system capable of being fixed on a bone and including an anchor and an awl. The anchor comprises an inner side, an outer side and a perforation in connection therewith, and the anchor further includes an accommodating portion and a thread portion. The accommodating portion and the thread portion are disposed at the inner side and the outer side of the anchor, respectively. One end of the awl penetrates the accommodating portion and protrudes from the perforation, and the shape of the cross section of the awl is non-circular. The awl is rotatable to drive the anchor, so that the anchor is screwed into the bone with the thread portion. The present disclosure provides a rotating awl with a non-circular shape in cross section to enlarge a pilot hole when drilling the bone, so that the anchor can be screwed into the bone while drilling.

### CROSS-REFERENCE

This application claims the priority benefit of Republic of China (Taiwan) Patent Application No. 109114001, filed on Apr. 27, 2020, the entirety of which application is incorporated herein by reference.

### TECHNICAL FIELD

The invention relates to an anchor implantation system, in particular to an anchor implantation system for medical surgery.

### DESCRIPTION OF RELATED ART

An anchor is a surgical equipment used to stitch soft tissues such as ligaments. The stitch procedure comprises a step of firstly securing one end of a stitch thread to a bone by means of an anchor, then stitching the soft tissue with the stitching thread, and finally uses another anchor to tighten and secure the other end of the stitching thread.

In order to implant an anchor into a bone, it is a common practice to drill the bone to form a pilot hole inside the bone by using an instrument having a bigger diameter, and then punch the anchor into the pilot hole. However, there are some problems with the above-mentioned practice.

First of all, with the progress of medical technology, a variety ofnew minimally invasive surgeries have emerged. Most of these minimally invasive surgeries are assisted with endoscopes, so that the viewing field of a surgeon is subject to many restrictions. Therefore, in the situation of separately performing a drill and an implantation of anchors, it is often that the surgeon cannot find the original drilling site after replacing the instrument.

In addition, because the anchor is fixed by punching, it is necessary to drill out a larger pilot hole in advance, which is a greater damage to the tissue around the bone, and the firmness of the anchor is also poor. In terms of surgical operations, the steps to replace the instrument also make the operation more cumbersome and time-consuming.

### SUMMARY OF THIS INVENTION

In order to improve the above problems of the conventional method, the present invention proposes an anchor implantation system, which can complete the process of drilling, implanting anchor and fixing anchor in one operation. It not only effectively reduces a mistake rate in surgery, but also saves time and enhances the effectiveness of surgery.

According to one aspect of the present invention, an anchor implantation system that can be fixed on the bone is provided. The anchor implantation system comprises an anchor and an awl. The anchor has an inner side, an outer side, and a perforation communicating the inner side and outer side. The anchor includes an accommodating portion and a thread portion. The accommodating portion is located at the inner side and the thread portion is located at the outer side. The awl penetrates the accommodating portion and has one end protruding from the perforation. The awl has a non-circular shape in cross section, and is rotatable to drive the anchor to screw into the bone with the action of the thread portion

Since the cross section of the awl according to one aspect of this invention is non-circular, it can not only be used to drive the anchor to rotate, but also expand a radius of a hole cut by the awl during rotation, which in turn create a pilot hole in the bone sufficient to allow the thread portion being screwed into the bone directly. In this way, the surgeon can directly screw the anchor into the bone after determining a proper surgery site, which in turn eliminates a need to drill a hole with other instruments before implanting the anchor.

In other aspects, the shape of the awl in cross section may be a triangle, a square, a pentagon, or a hexagon, and is not limited thereto. The awl can have a tip end that has a tapered shape and can be punched into the bone.

The thread portion may have a taper of 2 to 4 degrees. In a better embodiment, the taper can be 2.5 to 3.5 degrees, the best is 3 degrees. The material of the anchor can be polyether ketone (PEEK).

According to another aspect of the present invention, an anchor implantation system is provided, which comprises the anchor and the awl mentioned above in accordance with one aspect of the present invention, and a driving mechanism. The technical features of the anchor and the awl, as described previously, are not repeated again. The driving mechanism includes a driving shaft and an operating unit. The driving shaft connects another end of the awl and has a driving part. The operating unit connects the driving shaft and includes a guider, which connects the driving part to drive a rotation of the driving shaft. When the operating unit is driven and rotated, the driving part is guided by the guider, causing the driving shaft to move axially in relative to the anchor.

The features in this aspect of the present invention resides in that the awl can be controlled by the driving mechanism and axially retracted. In other words, when a pre-drill hole in the bone is required, the awl can be extended out using the operating unit and used as a drilling device. After the drilling is completed, the awl returns to its initial position and can be rotated into the bone along with the anchor.

By means of the above manner, the present invention may also be applied to a surgery with a pre-drilling, and can also complete the anchor implantation by one operation.

In the above manner, the guider can be a pin, while the driving part can be a groove. The groove has a spiral shape and recessed on the surface of the driving shaft. In addition, the driving shaft may have a positioning piece and the driving mechanism may include a grip part. The grip part is in the shape of hollow shell and can accommodate the driving shaft for its pass through. The grip part includes a limiting part for constraining the positioning piece, so that the grip part and the driving shaft is positioned in relative to each other.

When the limiting part and the positioning piece are constrained to each other, the grip part and the driving shaft cannot rotate in relative to each other. In this way, the operator can hold the grip part of the driving mechanism to operate the awl and screw the anchor into the bone.

The limiting part may be formed on the inner wall of the grip part, and the positioning piece may be a piece of block and can be accommodated in the limiting part.

In addition, the anchor can have a hole and the awl can have a concave groove for the pass of a stitching thread. The positions of the hole and the groove correspond to each other so that the stitching thread can get in from the groove and get out from the hole.

### BRIEF DESCRIPTION OF THE DRAWINGS

The invention will be described with reference to the following drawings, wherein like numerals refer to like elements, and wherein:

FIG. 1 is an explosive diagram of the anchor implantation system according to one embodiment of the present invention;

FIG. 2 is a partial cutaway diagram of the anchor implantation system shown in FIG. 1;

FIG. 3A is an assemble schematic diagram of the anchor implantation system shown in FIG. 1;

FIG. 3B is a front view of the anchor implantation system shown in FIG. 1;

FIGS. 4A to 4D show an operational process of the anchor implantation system shown in FIG. 1;

FIG. 5 is an explosive view of the anchor implantation system according to another embodiment of the present invention;

FIGS. 6A and 6B are schematic diagrams of an operating unit of the anchor implantation system shown in FIG. 5;

FIGS. 6C to 6E are schematic diagrams of a driving shaft of the anchor implantation system shown in FIG. 5;

FIGS. 6F and 6G are schematic diagrams showing a draw-back situation of the anchor implantation system shown in FIG. 5;

FIG. 7A is a cutaway diagram of a grip part of the anchor implantation system shown in FIG. 5;

FIGS. 7B and 7C are schematic diagrams showing a draw-back situation of the anchor implantation system shown in FIG. 5;

FIG. 8A is a schematic diagram of a thread-pass groove in the awl and a hole in the anchor that are included in the anchor implantation system shown in FIG. 5; and

FIGS. 8B to 8F are schematic diagrams showing an operating process of the anchor implantation system shown in FIG. 5.

### DETAILED DESCRIPTION OF EMBODIMENTS

In the following, with reference to accompanying drawings of embodiments of the invention, technical solutions in the embodiments of the invention will be clearly and completely described. Apparently, the embodiments of the invention described below only are a part of embodiments of the invention, but not all embodiments. Based on the described embodiments of the invention, all other embodiments obtained by ordinary skill in the art without creative effort belong to the scope of protection of the invention.

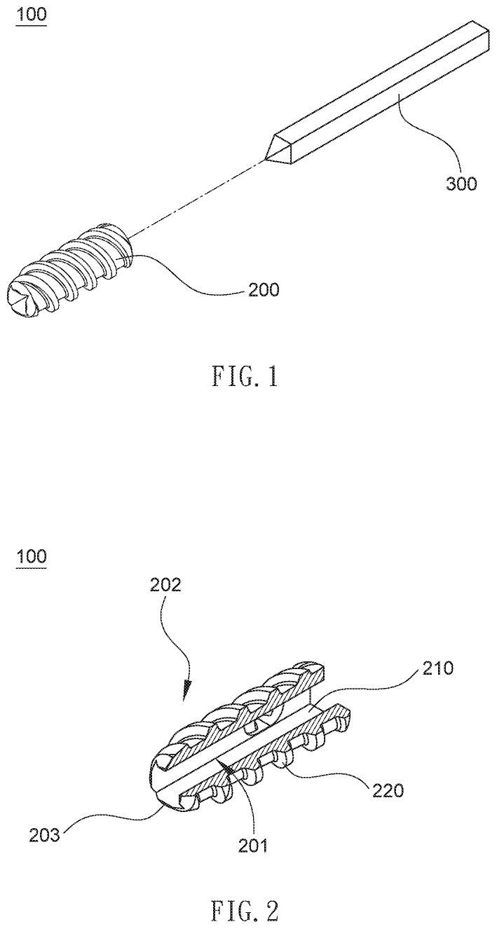

Referring to FIG. 1 and FIG. 2, an anchor implantation system 100 according to a first embodiment of the present invention includes an anchor 200 and an awl 300. The anchor 200 is in the shape of a thin shell and has an inner side 201 and an outer side 202. A front end of the anchor 200 is provided with a perforation 203 communicating the inner side 201 and the outer side 202. The inner side 201 of the anchor 200 is provided with an accommodating portion 210 for accommodating the awl 300. The outer side 202 of the anchor 200 is provided with a thread portion 220.

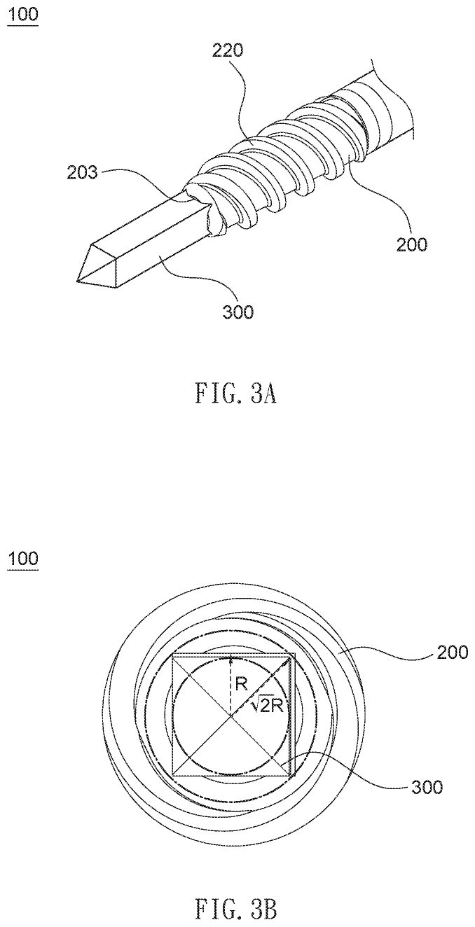

Referring to FIGS. 3A and 3B, the awl 300 passes through or penetrates the accommodating portion 210 of the anchor 200, and protrudes from the perforation 203 with its front end. The cross section of the awl 300 has a non-circular shape. As shown in FIG. 3B, R is a radius that is cut by of a general instrument, while a radius cut by the awl 300 is √R. In other words, the cross-section area of a pilot hole cut by the awl 300 increases one time in comparison to an instrument having a circular shape in cross section. It is noted that although the awl 300 in this embodiment can cut out a pilot hole having a larger cross-section area, the cross-section width of the awl 300 is still 2R. Therefore, the size of the anchor 200 is not necessary to be increased accordingly, which is good for the anchor to be screwed into the pilot hole.

In addition, although the cross section of the awl 300 shown here as an example is in the shape of a square, this should not constitute a limitation to the present invention. For example, the cross section of the awl 300 could be in the shape of a triangle, a pentagon, a hexagon, or others.

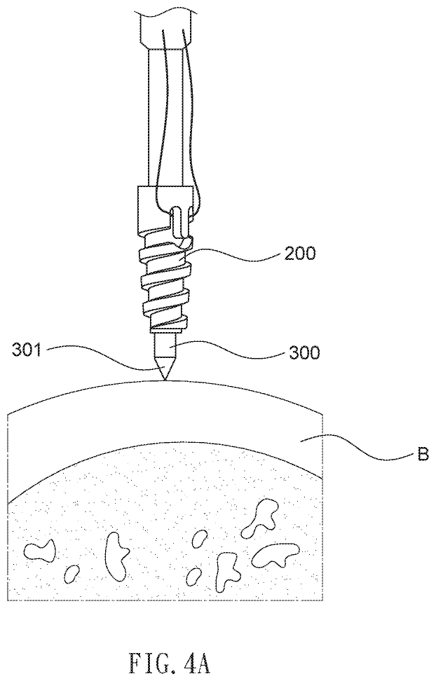

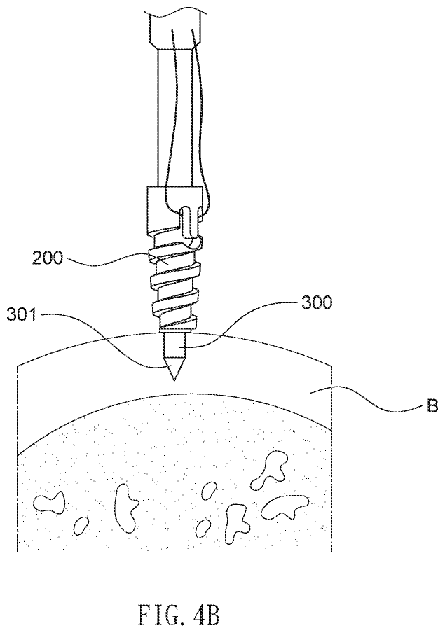

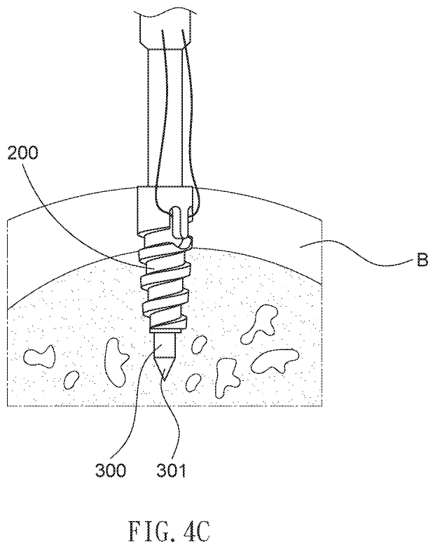

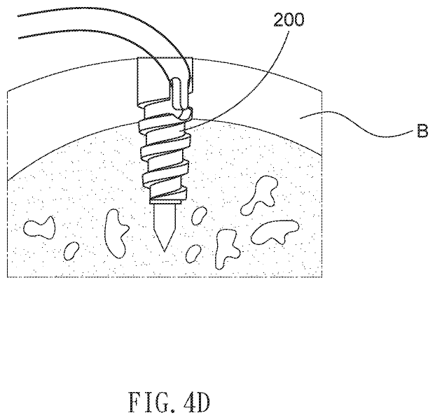

The FIGS. 4A to 4D show an operating process of the anchor implantation system 100. As shown in FIG. 4A, a surgeon can directly put the anchor implantation system 100 into a surgery site through a cannula and does not have to drill a polit hole with other instruments in advance.

As shown in FIG. 4B, the front end of the awl 300 is provided with a tip end having a tapered shape, which can be directly punched into or screened into a bone B when a surgeon has decided the surgery site of the anchor 200.

As shown in FIG. 4C, since the awl 300 has a non-circular shape in cross section, the anchor 200 can be rotated along with the awl 300 when the surgeon rotates the awl 300, causing the anchor 200 to be screwed into the bone B under the action of the thread portion 220.

As shown in FIG. 4D, after the anchor 200 is screwed into a predetermined depth, the surgeon can then draw the awl 300 out and proceed with a stitching surgery on soft tissue in the following step.

As mentioned above, the anchor implantation system 100 according to the embodiment of this invention integrates the awl 300 and the anchor 200, and the awl 300 can force the anchor 200 to screw into the bone B at the same time of drilling out a polit hole. In this way, it does not only omit the process of pre-drilling the pilot hole by the surgeon, but also simplify the operation of fixing the anchor 200. The fixing effect of the anchor 200 by this way is even better than conventional manner.

The thread portion 220 described above may have a taper of 2 to 4 degrees. In a better embodiment, the taper of the thread portion 220 can be 2.5 to 3.5 degrees, the best thereof is 3 degrees. The material of the anchor 200 can be polyether ketone (PEEK), or other materials that are compatible with the bone B.

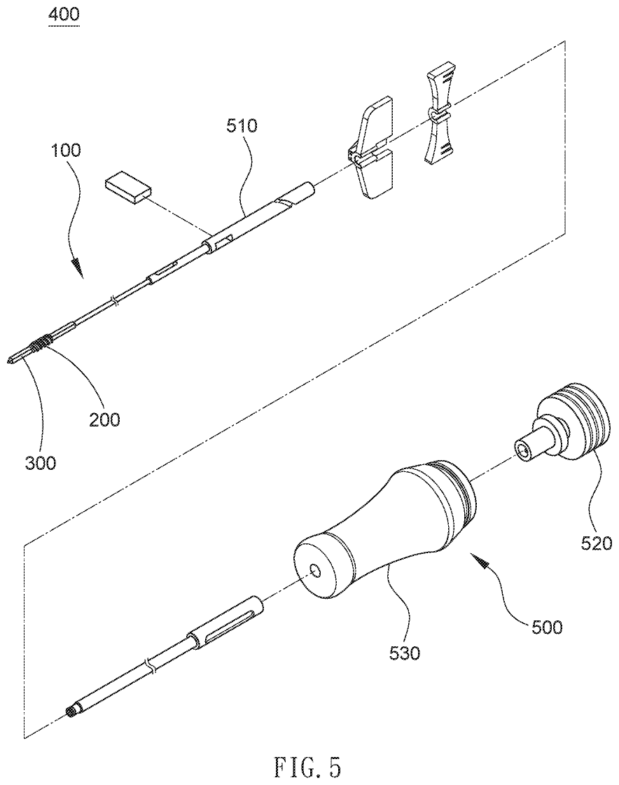

Referring to FIG. 5, it discloses an anchor implantation system 400 according to another embodiment of the present invention, which comprises the anchor implantation system 100 mentioned above and a driving mechanism 500. The driving mechanism 500 comprises a driving shaft 510 and an operating unit 520. The driving shaft 510 connects another end of the awl 300 of the anchor implantation system 100. The operating unit 520 connects the driving shaft 510.

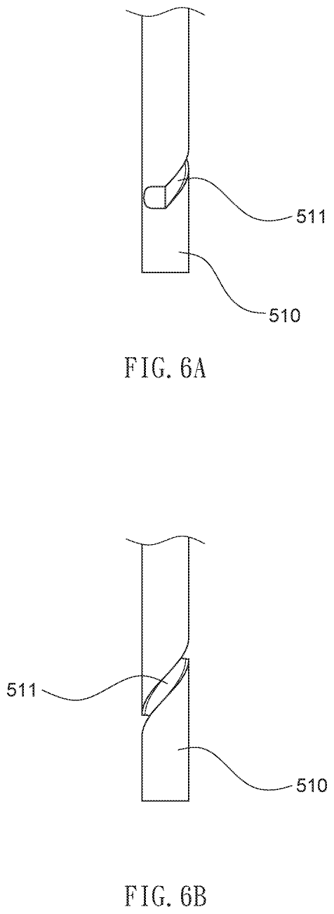

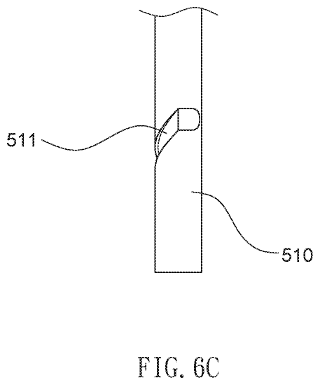

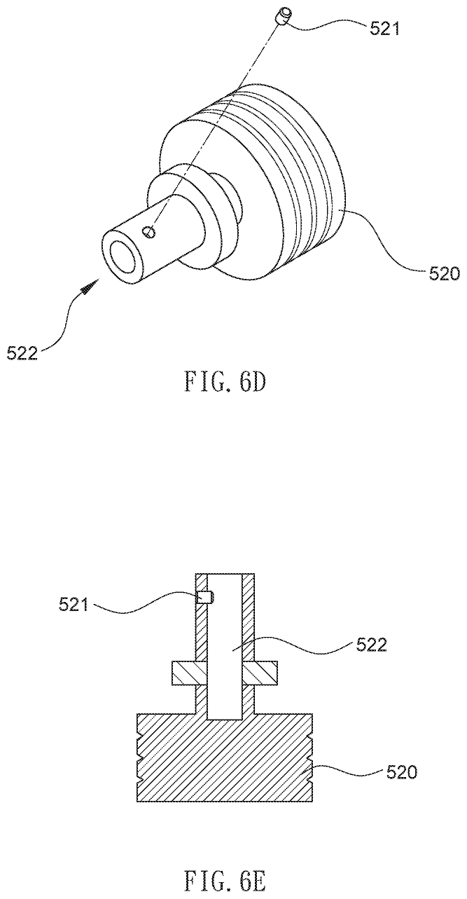

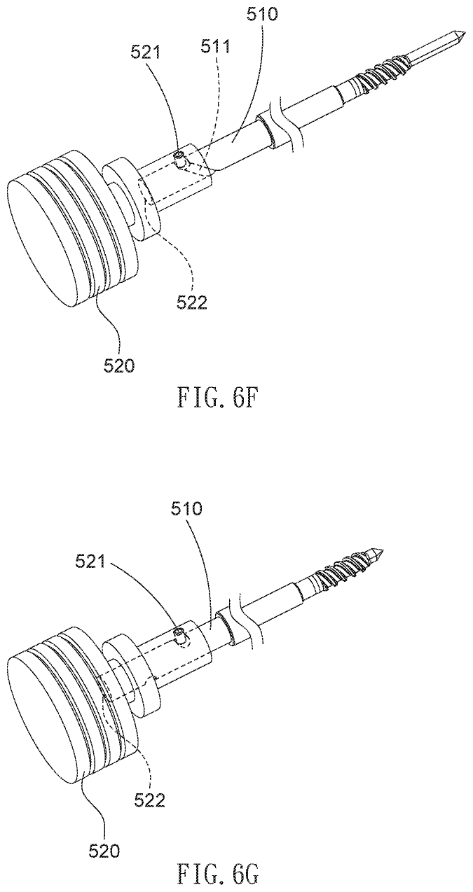

Referring to FIGS. 6A to 6C, the driving shaft 510 has a driving part 511. The driving part 511 is a spiral groove, and is recessed on the surface of the driving shaft 510. Continually referring to FIGS. 6D and 6E, the operating unit 520 comprises a guider 521, which is a pin fixed in the operating unit 520. As shown in FIG. 6E, part of the guider 521 penetrates into a connecting section 522 inside a wall of the operating unit 520.

Further referring to FIG. 6F, the driving shaft 510 is inserted into the connecting section 522 and the driving part 511 fits the guider 521. When the operating unit 520 rotates, the driving part 511 is guided by the guider 521, causing the driving shaft 510 to rotate together. In addition, since the driving part 511 is a spiral groove, in accompany of the rotation of the driving shaft 510, the driving shaft 510 also moves axially in relative to the anchor 200. As shown in FIG. 6G, when the guider 521 allocates at another end of the driving part 511, the driving shaft 510 reaches a bottom space of the connecting section 522. Meanwhile, the awl 300 connected to the driving shaft 510 also draws back in relative to the anchor 200.

According to the embodiment mentioned above, the awl 300 can be used to drill out a polit hole in the bone B first, and then be drawn back to its initial position by using the operating unit 520. By means of continually rotating the awl 300, the anchor 200 could be screwed into the bone B.

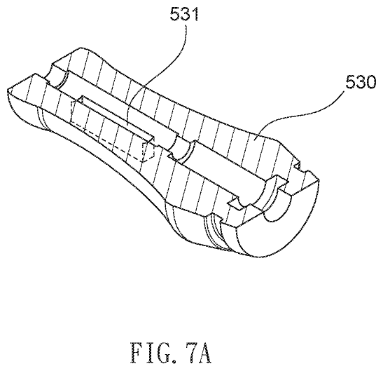

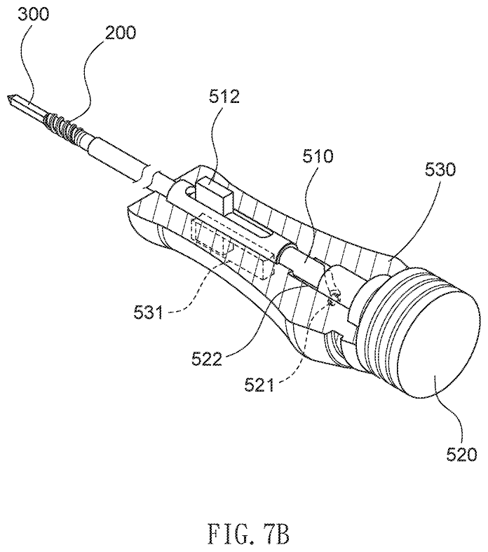

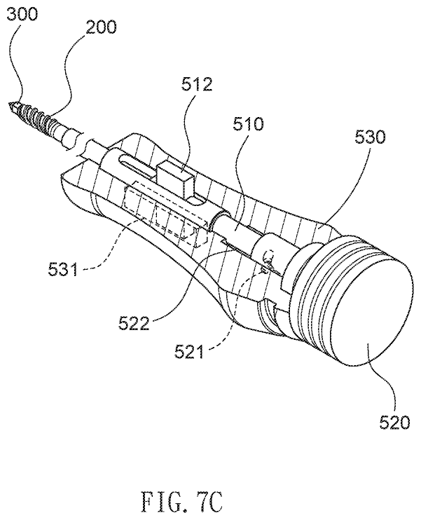

Referring to FIGS. 7A to 7C, FIG. 7A indicates that the driving mechanism 500 further comprises a grip part 530. The grip part 530 is in the shape of a hollow shell and can accommodate the driving shaft 510. The grip part 530 comprises a limiting part 531. As shown in FIGS. 7B and 7C, the driving shaft 510 also has a positioning piece 512. The driving shaft 510 is connected to the operating unit 520, and the operating unit 520 is adjacent to the grip part 530. In this embodiment, the positioning piece 512 is a block body, while the limiting part 531 is a chamber formed in an inner wall of the grip part 530.

In FIGS. 7B and 7C, the positioning piece 512 is a cuboid, and the limiting part 531 can accommodate the positioning piece 512. Therefore, the positioning piece 512 is constrained by the limiting part 531 such that the grip part 530, the operating unit 520 and the driving shaft 510 are fixed relatively in terms of positions. Thus, when the operating unit 520 or the grip part 530 is rotated, the driving shaft 510 is also rotated concurrently. During this process, the positioning piece 512 moves linearly in relative to the limiting part 531.

As shown in FIG. 7C, when the awl 300 draws back to a proper position, the surgeon can in turn rotate the operating unit 520 or the grip part 530 to implant the anchor 200 into the bone B.

It should be noted particularly that the grip part 530 is only to provide the operator a convenient way as the use of hand tools, and is not a necessary requirement to implement the present invention. In terms of having not the grip part 530, the operator can still rotate the driving shaft 510 with the operating unit 520 alone and screw the anchor 200 into the bone B.

Similarly, the specific configuration of the positioning piece 512 and the limiting part 531 can also be changed, as long as such a change can achieve the function of constraining a rotation between each other. Therefore, any changes are all applied to the present invention.

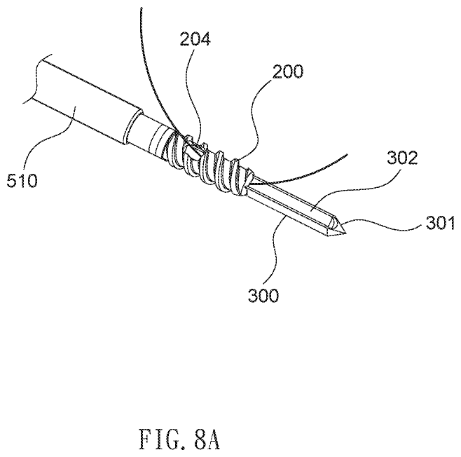

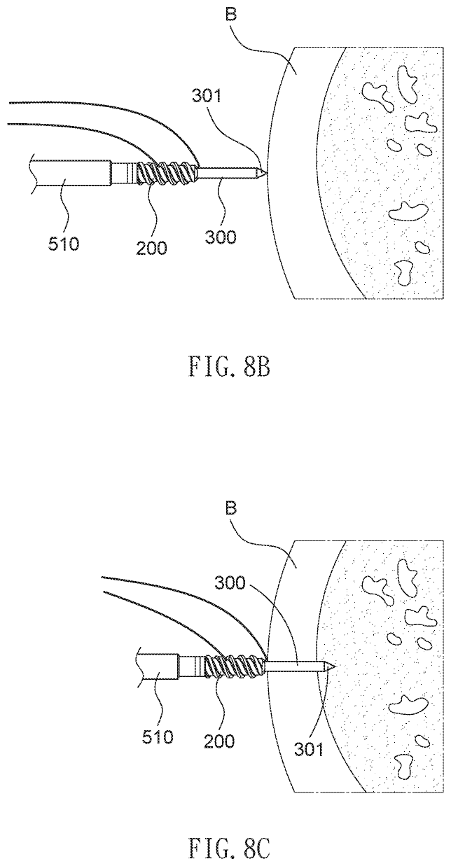

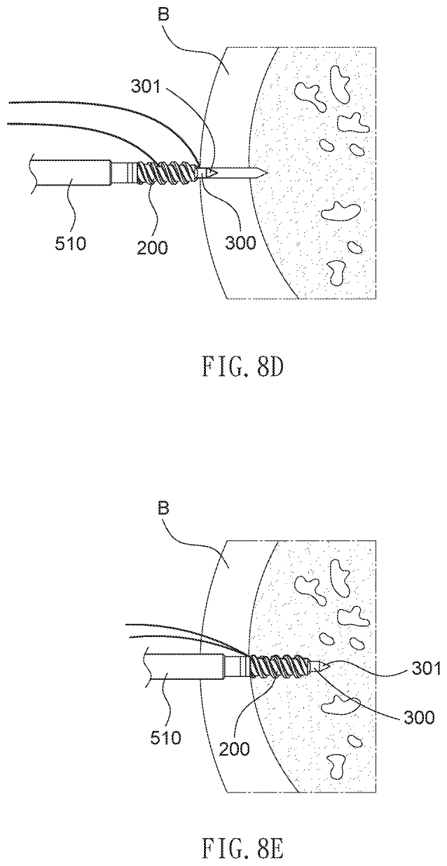

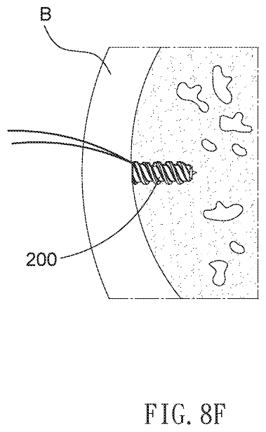

Continually referring to FIG. 8A, according to another embodiment, the anchor 200 has a hole 204, while the awl 300 has a thread-pass groove 302. The hole 204 and the thread-pass groove 302 have corresponding positions so as to permit a stitching thread pass through.

Referring to FIGS. 8B and 8C, as described in the first embodiment, the anchor implantation system 400 can be extended into a cannula to directly implant the anchor 200, without the need of drilling a pilot hole in the bone B by other instruments in advance. That is, the awl 300 is directly punched into the bone B after an implantation site of the anchor 200 is decided. Since the cross section of the awl 300 is non-circular, a rotation of the awl 300 will cut the bone B, and thus enlarges a diameter of the pilot hole.

Referring to FIG. 8D, the guider 521 drives the driving part 511 to draw the driving shaft 510 and the awl 300 back axially when a surgeon rotates the operating unit 520.

Continually referring to FIG. 8E, in a state of having drawn the awl 300 back, the surgeon can continue to rotate the operating unit 520 or the grip part 530, so as to screw the awl 300 and the anchor 200 together into the bone B. It is noted that since the awl 300 has been drawn back before the anchor 200 is implanted, the problem of twining a stitching thread on the awl 300 inside a sponge bone tissue is avoided. Therefore, when the anchor 200 is rotated into a proper depth, the awl 300 can be smoothly drawn out from the anchor 200.

As shown in FIG. 8F, when the anchor 200 is screwed into the bone B, the stitching thread located between the thread portion 220 and the bone B is also twisted and tightened which thus simultaneously completes a stitch of soft tissue.

As can be seen from the above embodiments, the present invention provides at least the following beneficial effects. First, the present invention uses an awl having a non-circular cross section to enlarge a cross-section area of the pilot hole, the anchor can be easily screwed into the bone and reach a good fixing effect. Second, the present invention integrates the awl and the anchor, and thus only need one single operating step to complete an implantation of the anchor. It does not only save time, but also reduce the difficulty of a surgery. Third, by means of incorporating the driving mechanism, the present invention can also support a process of drilling a polit hole first, which also omits a need to replace instruments after drilling, and thus can similarly obtain safety and efficiency.

The present invention has been disclosed in the above with a better embodiment, but the person familiar with this technology should understand that the embodiment is only used to illustrate the present invention, and should not be interpreted as limiting the scope of the present invention. Any change or displacement of the equivalent disclosed by the above embodiments shall be deemed to be covered within the scope of the present invention. Therefore, the scope of protection of the present invention shall be defined by the appended claims of this application for a patent.

What is claimed is:

1. An anchor implantation system that can be fixed to a bone, comprising:
an anchor having an inner side, an outer side, and a perforation communicating the inner side and the outer side, and including:
an accommodating portion located at the inner side; and
a thread portion located at the outer side; and
an awl, penetrating the accommodating portion with one end protruding from the perforation, having a non-circular shape in cross section, and being rotatable to drive the anchor to screw into the bone under the action of the thread portion.

2. The anchor implantation system as claim 1, wherein the cross section of the awl is in the shape of a triangle, a square, a pentagon, or a hexagon.

3. The anchor implantation system as claim 1, wherein the awl is provided with a tip end having a tapered shape for being punched into the bone.

4. The anchor implantation system as claim 1, wherein the material of the anchor is polyether ketone (PEEK).

5. The anchor implantation system as claim 1, wherein the thread portion has a taper of 2 to 4 degrees.

6. The anchor implantation system as claim 5, wherein the thread portion has a taper of 2.5 to 3.5 degrees.

7. An anchor implantation system, comprising:
an anchor implantation system as defined in claim 1; and
a driving mechanism that includes:
a driving shaft, which connects another end of the awl and has a driving part; and
an operating unit that connects the driving shaft and includes:
a guider, connecting the driving part to drive the driving part rotated;
wherein, when the driving shaft is driven and rotated, the driving part is guided by the guider, causing the driving shaft to move axially in relative to the anchor.

8. The anchor implantation system as claim 7, wherein the guider is a pin, and the driving part is a groove.

9. The anchor implantation system as claim 8, wherein the groove has a spiral shape and recessed on the surface of the driving shaft.

10. The anchor implantation system as claim 7, wherein the driving shaft comprises a positioning piece, and the driving mechanism further comprises:
a grip part, which is in the shape of a hollow shell being able to accommodate the driving part, and includes a limiting part; and
wherein the positioning piece and the limiting part are constrained by each other, so that the grip part is relatively positioned to the driving shaft.

11. The anchor implantation system as claim 10, wherein the limiting part is formed in an inner wall of the grip part.

12. The anchor implantation system as claim 11, wherein the positioning piece is a piece of block, and can be accommodated into the limiting part.

13. The anchor implantation system as claim 7, wherein the anchor has a hole, the awl has a thread-pass groove, and the position of the hole corresponds to the position of the thread-pass groove.
